(19) United States
(12) Patent Application Publication
(10) Pub. No.: US 2022/0000724 A1
(43) Pub. Date: Jan. 6, 2022

(54) Coating Compositions

(71) Applicant: THEUNSEEN LIMITED, London (GB)

(72) Inventors: Lauren Bowker, London (GB); Louise Anderson, London (GB)

(21) Appl. No.: 17/297,614

(22) PCT Filed: Nov. 27, 2019
(86) PCT No.: PCT/EP2019/082684

§ 371 Date: May 27, 2021

(30) Foreign Application Priority Data
Nov. 27, 2018 (GB) 1819323.5

(51) Int. Cl. A61K 8/02; A61Q 3/02

(52) U.S. Cl. CPC A61K 8/0295; A61Q 3/02; A61K 8/87; A61K 2800/884; A61K 2800/438

(57) ABSTRACT

A coating product comprising an aqueous chromic composition comprising a colour-change component, such as a liquid crystal and a polymer binder dispersed in an aqueous solution. The coating product also contains a colourant which may be dispersed or dissolved in the aqueous composition is provided as a separate aqueous solution comprising the colourant to provide an underlying layer.

This invention relates to a coating composition, particularly to a liquid crystal composition for coating a substrate particularly to nails and artificial nails and existing coatings on nails. The invention is particularly concerned with an aqueous composition comprising a liquid crystal component which provides a colour-change effect and a black dye or pigment.

A wide range of decorative cosmetics for coating substrates such as keratinous living substrates, for example nails, skin, lips and the like and non-living substrates such as artificial nails, for example acrylic nails and nail extensions are available in the personal care market to provide aesthetic and/or functional effects. Such compositions may be aqueous-based or solvent-based depending on the particular type of product and typically must comply with toxicology and other safety and testing standards and protocols.

Commercially formulated nail products are typically in liquid or gel form and comprise a number of components to provide a desirable combination of characteristics such as ease of application and evenness of coating, durability and resistance to chipping and washing, vibrant colour, reasonable drying time and non toxic. Nail coatings typically comprise organic solvents, a colour pigment and a film forming component and upon deposition, the solvents evaporate to leave a dried durable coating and may include rheology modifiers, plasticisers, colour additives, for example pearl agents, mica and the like, viscosity modifiers, UV additives and other components.

Nail compositions having chromic components providing a colour change effect are known. Chromic components change between a first and second state which may both be coloured or white, or one coloured or white and the other colourless, in response to a change in an external condition such as application of energy for example through a change in temperature or pressure or incidence of light. Chromic components which change colour with a change in temperature are known as thermochromic, with pressure as piezochromic, with water, hydrochromic and, with light, as photochromic. Chromic components which change colour or opacity between different states or in response to a stimulus are referred to herein as “colour change components”. Colour change in chromic compositions may be provided by different types of materials including leuco-colourants and liquid crystals.

Thermochromic compositions reversibly change colour or change between different coloured states or a coloured and colourless state when subjected to a change in temperature of a sufficient magnitude. Typically, a change in in temperature will lead to a change in colour of the colour-change material. With an increase or decrease in temperature, the colour-change material will typically retain colour until a maximum or minimum temperature for retention of the complete coloured state is reached. The composition will then progressively change colour or become colourless as the temperature increases or decreases until it has completely changed colour. As the thermochromic composition cools it will reach a temperature below which the colour changes, disappears or reappears. Thermochromic materials, having standard colour change schemes and bandwidths, that is a difference in temperature at which colour change occurs from a first to a second colour and in reverse from the second colour to the first, are commercially available, for example from LCR Hallcrest.

Commercially available nail coating products are typically based on organic solvents. Aqueous-based nail coating compositions are known but commercialisation has proved difficult. In solvent-based nail coating compositions, nitrocellulose is typically employed as a film-forming component together with volatile solvents such as ethyl acetate and butyl acetate and toluene. Evaporation of the solvent desirably provides rapid drying after application of the composition. Whilst nail compositions based on organic solvents may be tailored according to user requirements, they may also preclude the use of components which are incompatible with such organic solvents. Furthermore, organic solvents may be toxic, flammable, have a characteristic and undesirable aroma and the production methods for producing such products may have environmental drawbacks.

In chromic cosmetic products, colour-change may be provided by a leuco dye although certain solvents may affect the colour-change properties of such materials and careful formulation is required. The colour-change effect and the conditions under which it occurs, for example a particular temperature range may be controlled by modifying the leuco dye composition in a manner known to those skilled in that field.

Liquid crystals typically provide more intense colours than leuco dyes. However, organic solvents are known to be detrimental to the colour-change properties of liquid crystals, particularly to unencapsulated liquid crystals where the solvent may adversely interact with the liquid crystal and therefore render them unsuitable for use in organic solvent-based products. Encapsulating the liquid crystal provides some protection, however organic solvents can still penetrate through the capsule and interact with the liquid crystal, again rendering them unsuitable for use in an organic solvent.

The colour intensity or brightness of liquid crystals may be reduced or impaired where wavelengths of light other than that reflected by the liquid crystal are also reflected. Use of a conventional, solvent-based, black nail polish as an underlying layer for a liquid crystal nail product is known. However, the solvent in an underlying layer may migrate into an overlying layer to some degree and adversely affect solvent-sensitive components in an overlying layer.

We have now found that a colour-change nail product having intense colour without the liquid crystal being impaired by providing a product which has a liquid crystal and a colourant, in combination or in separate layers in an aqueous solvent.

In a first aspect the invention provides a coating product comprising an aqueous chromic composition comprising a colour-change component and a binder dispersed in an aqueous solution and wherein the coating product:

i) further comprises a colourant dispersed or dissolved in the aqueous composition; or

ii) comprises a separate aqueous solution comprising a colourant adapted for use with the aqueous chromic composition to provide an underlying layer comprising colourant underlying the colour-change component.

The coating product is suitable for application to natural nails and artificial nails.

The colour-change component suitably comprises a liquid crystal. Preferably, the coating product comprises:
i) an aqueous chromic composition comprising a liquid crystal and a binder dispersed in an aqueous solution; and
iii) a separate aqueous solution comprising a colourant adapted for use with the aqueous chromic composition to provide an underlying layer comprising colourant underlying the colour-change component.

Known nail varnishes typically comprise an organic solvent or have an organic solvent base which carries the functional components such as the colourant. Liquid crystal, particularly encapsulated liquid crystal, is typically not stable in organic solvent. Preferably, the aqueous chromic composition is substantially free of organic solvent. Preferably, the separate aqueous solution is substantially free of organic solvent.

In a second aspect the invention provides a method of providing a coating which comprises applying an aqueous composition according to the invention to a substrate to be coated optionally after an underlying layer has been applied to the substrate and dried, the aqueous chromic composition comprising a colour-change component and a binder dispersed in an aqueous solution and wherein an underlying layer has not been applied, the coating product further comprises a colourant dispersed or dissolved in the aqueous composition.

In a further aspect, the invention provides a dried or cured coating comprising a colour-change component dispersed in a continuous phase comprising a dried or cured binder is derived from a dried or cured oligomer and/or, a dried or cured monomer, and an initiator, wherein the colour-change component is dispersed in the said continuous phase.

Colour-change in the chromic composition may be provided by different types of colour-change components including leuco-colourants, particularly thermochromic leuco-colourants which are responsive to changes in temperature, photochromic leuco colourants which are responsive to UV or other light and hydrochromic leuco colourants which are responsive to contact with water, vapochromic leuco-colourants, which are responsive to the presence of certain gases or vapours and may be used to detect the presence of pollution or other environmental agents, solvatochromic leuco-colourants which respond to changes in polarity, ionochromic leuco-colourants which respond to changes in ion concentration, halochromic leuco-colourants which can detect changes in pH, for example changes in the pH of the substrate of the nail or the adjoining skin.

The colour-change component may be tailored to provide a colour-change after a period of time has elapsed rather than in response to a particular stimulus.

The colour-change component may be selected so as to provide a single colour above a pre-determined temperature or below a pre-determined temperature.

The colour change component may undergo one or more successive colour changes. The colour change may be reversible or irreversible. The colour change may be in response to the application, presence or change of a stimulus, for example heat, light, pressure, water, certain gases, polarity, ion concentration and pH or which changes colour over time without any other stimulus whether in seconds or days, for example by tailoring the bandwidth such that component is in a particular state presenting a different colour for a shorter or longer period of time.

The thermochromic liquid crystal is preferably encapsulated or may be unsealed or unencapsulated. A polymer-dispersed liquid crystal comprises a discrete phase comprising the liquid crystal dispersed in a continuous polymer phase in which the liquid crystal is present in droplets, typically of a micron scale, for example from 0.01 to 50 microns. Unsealed liquid crystals are less preferred due to difficulties in incorporating them in an aqueous composition.

In a preferred embodiment, the colour-change component comprises encapsulated liquid crystal and desirably consists of only encapsulated liquid crystal.

The coating product is especially suitable for coating keratinous material directly or indirectly, particularly nails and for coating artificial, non-keratinous nails and nail extensions, for example acrylic nails. The coating may be applied to an existing coating, for example a base coat, a gel coat, a shellac coat and polished nails and to uncoated nails.

Advantageously, the product of the invention enables a striking and intense colour or chromic effect to be achieved without the detrimental aspects of using an organic-solvent based product by enabling the effective use of liquid crystal colour-change components in combination with a colourant providing a fixed or constant colour. The aqueous-based product allows ready removal or cleaning in the event of spillage or mis-application using water or water-based cleaning products without the need to employ solvent-based products or varnish removers.

The coating product may be tailored to provide a desired visual appearance for particular circumstances or moods and adapted to provide a faster or slower colour change with single or multiple colour change within a bandwidth of temperatures or other external parameters.

The encapsulate suitably comprises a known encapsulation material which is compatible with the liquid crystal. In one embodiment, the liquid crystal chromic component is suitably encapsulated in a wall comprising a gelatin and gum arabic mixture, for example gum arabic and/or a gluteraldehyde cross-linked gelatin. Other encapsulate materials which are known or subsequently devised may be employed. Examples of other types of capsules include siloxane based capsules, thermoplastic polymer based capsules and thermoset polymer based capsules.

Suitable liquid crystals include cholesteric liquid crystals which are typically based on or comprise cholesterol or other sterol derived compounds including cholesteryl carbonate esters. Other suitable liquid crystals include non-sterol based compounds, for example phenyl benzoate esters.

The coating product may further comprise a leuco colourant in combination with the liquid crystal. Combinations of leuco colourant and liquid crystal are especially suitable for providing multiple, for example at least 3 colour changes. Combinations of colour-change materials are suitably mixed together.

Suitable encapsulated liquid crystals are available from a range of sources including LCR Hallcrest. Non-cholesterol based liquid crystals such as phenyl benzoate esters provide bright, strong colour effects with high reflectance, suitable for premium products. Cholesteric-type liquid crystals typically provide colour effects which are less intense than non-cholesteric liquid crystals but are of lower cost and may be suited to larger volume markets. A mixture of non-cholesteric and cholesteric liquid crystals may be employed to optimise product performance and cost. In a preferred embodiment, the liquid crystal suitably comprises only non-cholesteric liquid crystals.

The encapsulated colour-change component, preferably an encapsulated liquid crystal, is preferably present at a level of 0.5 to 90%, 5 to 70% and especially 5 to 60% by weight of the chromic composition. In some embodiments, the encapsulated colour change component may be present at a level of 5 to 50% and especially 6 to 30%, for example 12%, 16% and 20% by weight of the chromic composition.

Encapsulated liquid crystals are suitably incorporated into the product as a slurry of the microencapsulated liquid crystal in an aqueous slurry. Preferably the slurry comprises water, the encapsulate and a preservative. Suitably the encapsulated liquid crystal is present in the slurry at a level of 10 to 90%, preferably 15 to 60% and especially 20 to 50%, for example 40% by weight of the aqueous slurry. The preservative is suitably present at a level of 1% by weight of the aqueous slurry. Examples of suitable preservatives include germaben II, which is a mixture of diazolidinyl urea, methylparaben, propylparaben in propylene glycol. Use of any other compatible preservative or combination of preservatives can be used including but not limited to phenoxyethanol, benzyl alcohol, methylparaben, propylparaben and sodium benzoate.

The nail product also comprises a colourant which does not present a change in colour in normal usage. The colourant may comprise any material which presents a single colour in the conditions in which the composition is to be is used. A single colour may be provided by a colourant which does not change colour, for example a dye and/or a pigment or by a liquid colourant which is capable of colour change for example a liquid crystal or leuco colourant, but which colour change does not occur under the conditions of normal usage of the composition, known as a “static” colourant. The term “static” refers to a leuco colourant or a liquid crystal which does not change colour under the conditions of use. Thus while the colourant is capable of colour-change, it does not typically exhibit colour change under the conditions of use and shows a fixed colour.

The colourant suitably comprises a static liquid crystal, a dye which is aqueously soluble and/or a pigment which is a fixed colour and aqueously dispersible. Suitably, the colourant comprises a dye. Pigments may scatter light providing a “whitening” effect or haziness. A dye and/or pigment may accordingly be selected according to the intended desired effect. A pigment may provide a more singular colour change.

The term “singular” refers to a “two-tone” colour system, either a static colour or a marked colour change from colourless to coloured, or coloured to colourless, or from a first colour to a second colour, or a fluctuating colour between two colours as opposed to the traditional spectrum of colours, red, green and blue and transition between them typically observed with liquid crystal. For example a singular liquid crystal system may fluctuate between two colours such as green and blue without showing red. The term “singular” also refers to a single colour being observed above or below a certain pre-determined or tailored temperature. A singular colour liquid crystal suitably has a thicker encapsulate wall, for example 5 to 10 microns.

Haziness may be imparted intentionally to provide a visual effect by use of a pigment or by applying multiple layers of the liquid crystal or multiple layers of the binder such that reflected light is diffracted. The nail product may comprise the colourant and colour-change component as a single composition in which the colourant and colour-change component are intimately mixed or may comprise at least two separate aqueous compositions, a first aqueous composition comprising the colour-change component and a second aqueous composition comprising the colourant. In the latter case, the second composition will suitably be applied as an undercoat on the substrate and the colour-change component then applied on top of the undercoat, preferably after the undercoat has dried.

The colourant is a fixed colour and enables or enhances visual appearance or enhancement of the properties of the liquid crystal. The colourant may be any colour for example red, orange, yellow, green, blue, indigo, violet, brown or black. Darker colours are preferred as they provide a more intense colour change effect to the observer. Lighter colours may offer a pearlescent or opalescent effect which may be desirable in some cases. The colourant may be a dye or a pigment. Dyes are typically soluble in water and pigments are typically dispersible in water. Dyes are preferred as pigments may obscure reflectance from the liquid crystal and reduce colour intensity.

The colourant may be incorporated into the nail product in a neat form, as a powder or as a solution of the colourant at any desired level. The colourant solution may comprise 0.1 to 10% aqueous solution of the colourant or, if added as a powder, is suitably present at a level of 0.1 to 2.5%, preferably 0.5 to 2% for example 1%. The colourant solution or dispersion may be incorporated into the chromic composition at a level dependent on the concentration of the colourant solution and the desired final content of the colourant in the product. For example a 1% solution of the colourant may be included at a level of 1 to 20%, preferably 5 to 15% for example 10% by weight of the chromic composition.

Preferably, the colourant is black or blue, for example carbon black, In a preferred embodiment, the colourant comprises carbon black, iron oxide, mica, graphite synthetic melanin and polydopamine. pigment. Examples of suitable water-based black dye or pigments include products available under the trade name MINISO.

In a preferred embodiment, the invention provides an aqueous-based coating product comprising a single composition comprising a chromic composition comprising an encapsulated liquid crystal and a dark colourant, preferably a blue or black colourant in an aqueous solution or dispersion.

Advantageously, the single composition is easier to use and does not require a user to apply a colourant undercoat, wait for that to dry and then apply an overcoat containing the liquid crystal. In the single composition, a proportion of the colourant may overlie liquid crystal and thereby reduce the effective reflectance of the liquid crystal. In this embodiment, a greater level of liquid crystal may be employed to mitigate or avoid any reduction in brightness due to the presence of colourant in the single composition.

The binder suitably comprises a film-forming polymer which is soluble or dispersible in aqueous solution and aids an attractive aesthetic effect, in particular excellent surface reflection, providing intense colours, contrast between different colours and responsive colour change and adherence to the substrate and other desirable characteristics may be secured.

In a further aspect the invention provides a coating product comprising an aqueous solution which comprises an encapsulated liquid crystal and a binder dispersed in the aqueous solution and a dark colourant provided in the said aqueous solution or provided in a separate aqueous solution wherein the binder comprises at least one of a non-ionic, hydrophobically-modified ethoxylated urethane (HEUR), a polyurethane, a styrene/acrylate/ammonium methacrylate copolymer and/or an epoxy resin.

Suitably, the binder is selected from a non-ionic, hydrophobically-modified ethoxylated urethane (HEUR), an acrylic polymer, a styrene/acrylate copolymer, a styrene/acrylate/ammonium methacrylate copolymer, an epoxy resin and a polyurethane, for example polyurethane-2 is a copolymer of hexylene glycol, neopentyl glycol, adipic acid, saturated methylene diphenyldiisocyanate and dimethylolpropionic acid monomers. Examples of preferred binders include SYNTRAN 5620CG available from Interpolymer, CARFIL 9235, 9241 and 9240, available from Wanhua, polyurethane-2 dispersion

Preferably, the binder composition is transparent. Advantageously a transparent binder augments the colour effect from colourant, especially a liquid crystal.

The binder is preferably provided as an aqueous composition with 5 to 50% solids and provided at a level such that the binder is present in the chromic composition at a level of 30 to 80, preferably 40 to 75, especially 50 to 70% by weight of chromic composition.

The aqueous based coating product according to the invention may be applied directly to the nail or may be applied to an undercoat which has been applied to the nail. The undercoat suitably comprises an aqueous composition known in the art and any compositions commercially available in the field may be employed. Suitable examples include SOPHI by Piggi Paint nail polish, ACQUARELLA, SUNCOAT nail polish, LITTLE ONDINE'S nail polish, KEEKI PURE & SIMPLE nail polish, UNT peelable nail polish, Honeybee gardens Watercolours nail polish, Karma organic two in one base coat/top coat, Sundays base coat nail polish and Sundays hydrating base coat No. 1.

The aqueous based coating product according to the invention, once applied to the nail may be coated with an overcoat. The overcoat may provide an aesthetic or functional purpose or both, for example providing a protective coating for the aqueous based nail product. The overcoat suitably comprises an aqueous composition known in the art and any compositions commercially available in the field may be employed. Examples of suitable overcoats include SUNCOAT clear top coat nail polish, LITTLE ONDINE water-based matte top coat, Karma organic two in one base coat/top coat, Sundays top coat nail polish,

We have found that providing a top-coat composition containing a UV absorbent material provides improved product life once applied to the nails.

In a further aspect, the invention provides a coating product comprising an aqueous chromic composition comprising a colour-change component and a binder dispersed in an aqueous solution and wherein the coating product:

i) further comprises a colourant dispersed or dissolved in the aqueous composition; or

ii) comprises a separate aqueous solution comprising a colourant adapted for use with the aqueous chromic composition to provide an underlying layer comprising colourant underlying the colour-change component

and wherein the product further comprises a separate aqueous top-coat composition comprising a UV absorbing agent and water.

The coating product may accordingly comprise an aqueous colourant composition, a separate aqueous chromic composition and an aqueous top-coat composition which, in use are applied successively to the nail with each coat being dry before application of the next coat. The aqueous colourant composition and the aqueous chromic composition may be mixed together to provide a single composition for application of a coat containing the colour-change component and the colourant. The top-coat composition is then applied to the dried coat.

In yet a further aspect the invention provides a method of providing a coating which comprises applying an aqueous composition according to the invention to a substrate to be coated optionally after an underlying layer has been applied to the substrate and dried, the aqueous chromic composition comprising a colour-change component and a binder dispersed in an aqueous solution and wherein an underlying layer has not been applied, the coating product further comprises a colourant dispersed or dissolved in the aqueous composition, drying the aqueous chromic composition and applying to the dried surface of the chromic composition an aqueous top-coat composition comprising a UV absorber and drying the applied top-coat composition.

Preferably, the UV absorber is present at a level of 0.5 to 10%, and more preferably 1 to 5% by weight of the top-coat composition.

Any known UV absorbers may be employed and preferred examples include avobenzone, benzophenones and octyl methoxycinnamate.

In a further aspect, the invention provides the use of a combination of a black colourant and a chromic product comprising a liquid crystal in an aqueous nail coating composition to provide a colour-change effect with intense colour. The responsiveness of the colour change effect is suitably between 1 millisecond and 10 seconds. The change suitably occurs in response to a change in temperature, either through heating up or cooling down, via conduction, convection, or thermal radiation heating.

The coating product may be applied to a substrate by a film or coating forming process, by for example spraying, dyeing, screen-printing and the like. For nails, the coating product may be applied conventionally using a brush.

The coating product is compatible with the encapsulation wall material to ensure structural integrity for the encapsulate. Preferably, the coating product comprises water and is present at a level of 10 to 90%, more preferably 30 to 80%, for example 50% by weight of the coating product.

Upon application, the coating product dries and, to reduce the drying time, the solvent may comprise a minor amount of organic solvents, sufficient to aid drying but not to disrupt the liquid crystal. The product may comprise an alcohol, ketone or aldehyde, for example ethanol, isopropanol; and acetone. Suitably, any such organic solvents are present at a level such that the total organic solvent level is less than 20%, preferably less than 10% by weight of the coating product. Where the product comprises a separate liquid crystal composition and a separate black dye or pigment composition, the solvent may be present in either or both compositions.

The composition may comprise a coalescing aid to aid drying of the film. Examples of suitable coalescing aids include hexylene glycol, propylene glycol, glycerine, propylene glycol n-butyl ether and dipropylene glycol dibenzoate.

The nail product may be non-iridescent or iridescent according to the desired effect. Other appearances may be provided, for example pearlescent, gloss and fluorescent, particularly with incident light near the UV spectrum. Iridescence suitably provides scattering of incident light to provide a shimmering effect of multiple colours or a rainbow of colours and arises due to the nail product having suitable refractive characteristics. Advantageously, PDLC liquid crystals may provide iridescent qualities as well as a colour-change properties.

The coating product preferably comprises an abrasion additive to provide hardness and resistance to abrasion for the product once coated on the substrate. Suitably, the abrasion additive is selected from an oxidised polyethylene wax and a cross-linking agent which would cross link the binder. The abrasion additive may be difunctional or multi-functional so as to facilitate formation of cross-links which may impart improved hardness and/or abrasion resistance to the coating.

The nail product may comprise an optical brightener to promote colour intensity. Any known optical brighteners may be employed, for example stilbenes.

Optionally, the coating product also comprises a rheology modifier to aid dispersibility, flow and spreading of the composition on the nail during application. The rheology modifier will preferably increase the stability over time, preventing the formulation from separating, while allowing ease of application. Rheology modifiers known for use in personal care products and which are soluble or dispersible in aqueous solution may be employed in the binder composition. The rheology modifier may comprise a natural thickener, a synthetic thickener and/or a dispersant or surfactant. Any known thickeners suitable for use in the intended field of use of the composition may be employed.

Examples of natural thickeners include egg yolk, starch, cellulose, alginate, chitosan, agar, arrowroot, carrageenan, collagen, gelatin, guar gum, pectin and xantham gum. Examples of synthetic thickeners include alkali-swellable emulsions (ASEs), hydrophobically modified alkali-swellable emulsions (HASEs) and hydrophobically ethoxylated urethane resins (HEURs). Examples of preferred dispersants include polysorbate 20/80, glyceryl cocoate, caprylyl glucoside.

The rheology of the system can also be modified using powders, including mineral-derived options such as silica and kaolin (anhydrous aluminium silicate).

In one embodiment, the rheology modifier comprises a glycol, preferably selected from polyethylene glycol, polypropylene glycol, polybutylene glycol and a C₂ to C₆ alkylene glycol, for example PEG 32, hexylene glycol and propylene glycol. The rheology modifier, if present, is suitably present at a level of 0.1 to 20%, preferably 1 to 10% for example 7% by weight of the binder

The chromic composition may also contain a viscosity modifier to provide improved thixotropic (shear-thinning) properties and aid wetting. Advantageously, the composition may be thicker when stored and readily thinned upon application aiding storage stability improving performance upon application. Suitably the viscosity modifier comprises a hydrophobically modified associative thickener, for example CARFIL 9241.

Optionally, the binder also comprises a pH modifier and may act to provide a higher alkalinity or acidity. A pH modifier may be required depending on the polymer employed as the binder in order to ensure the fixative does not adversely disrupt the encapsulation wall of the colour-change composition. pH modifiers known for use in personal care products may be employed in the binder composition.

The pH modifier, if present, is suitably present at a level of 0.1 to 10%, preferably 0.5 to 5% for example 3% by weight of the binder.

The nail product may also comprise a preservative such as a natural or synthetic antimicrobial. Examples of natural antimicrobials include, caprylyl glycol, tea tree oil, potassium sorbate and sorbic acid. Examples of synthetic antimicrobials include phenoxyethanol, benzyl alcohol, diazolidinyl urea, sodium benzoate, potassium sorbate, methylparaben, propylparaben and gluconolactone.

The nail product may comprise a UV additive. Known UV additives or absorbers may be employed including tuitanium dioxide, zinc oxide, and avobenzone. Preferred UV additives include benzophenone-4 and phenylbenzimidazole sulfonic acid EUSOLEX 232 available from Merck.

The nail product may also comprise a plasticiser, with known commercially available plasticisers being suitable.

The nail product may also comprise a perfume, especially if the composition is intended for use on the human or animal body. Examples of suitable perfumes include peppermint oil, sweet orange essential oil, menthol, linalool, partum, methyl butyrate, citral, myrcene, limonene and eucalyptol.

As desired, the nail product may comprise a further colourant in addition to the colour-change component and the colourant.

Examples of suitable dyes include any dyes suitable for use in cosmetic products may be employed including the following used in combination or on their own; N,N-Bis(2-Hydroxyethyl)-2-Nitro-p-Phenylenediamine, 4-Amino-3-Nitrophenol, 4-Hydroxypropylamino-3-Nitrophenol, Hydroxyethyl-2-nitro-p-toluidine, HC Blue No. 12, 3-Nitro-p-Hydroxyethylaminophenol, 2-Amino-6-Chloro-4-Nitrophenol, Acid Red 33, HC Yellow No. 2, HC ORANGE NO. 1, HC YELLOW NO. 13, Basic Yellow 57, HC Red No. 3, Basic Red 76, Basic Red 51, Acid Red 92, Basic Yellow 87, Basic Orange 31, BASIC VIOLET 2, HC Blue No. 16, Acid Violet 43, Basic Brown 17 and dyes provided by Sensient® Cosmetic technologies; Arianor Jade Blue (HC Blue 15), phat black DC 9206 (mixture of CI61565, CI60725, basic brown 16, acid violet 43, basic red 76, CI 26100), Arianor flash deep black (Basic Blue 99, Basic Brown 16, Acid Violet 43, Basic Red 76, Basic Yellow 57, Basic Brown 17, Polyquaternium-37, Hydrolyzed Yeast Protein)

Examples of suitable pigments include any pigments suitable for use in cosmetic products may be employed including the following used in combination or on their own; Iron oxides, titanium oxides, synthetic mica (synthetic fluorphlogopite), carbon black, lakes, natural mica, silica, Covalumine fire red AS (CI 15850, Alumina, Triethoxycaprylylsilane), Noir W 699 (CI 20470, Sodium Sulfate), Covalumine astral blue (CI 42090, Alumina, Triethoxycaprylylsilane), Unipure white LC986 FSP (CI 77891, Perfluorooctyl Triethoxysilane, Polyperfluoromethylisopropyl Ether), Unipure red LC3071 (CI 15850, Aluminum Hydroxide), Unipure white LC987 GCA (CI 77891, Sodium Cocoyl Glutamate, Cystine, Lauroyl Arginine), Covarine white WN9787 (CI 77891, Aqua, Glycerin, Xanthan Gum, Sodium Citrate), Covapate Uniwhite LC 9781 (Ricinus Communis (Castor) Seed Oil, CI 77891, Polyhydroxystearic Acid), Unipure white LC981 SGP (CI 77891, Sodium Glycerophosphate), Vert covasol W7035 (CI 19140, CI 42090), unipure yellow LC182 ADT-C (CI 77492, Isopropyl Titanium Triisostearate, Bis-PEG-15 Dimethicone/IPDI Copolymer, PEG-2 Soyamine), unipure black LC989 ADT-C (CI 77499, Isopropyl Titanium Triisostearate, Bis-PEG-15 Dimethicone/IPDI Copolymer, PEG-2 Soyamine), unipure black LC988 FSP (CI 77499, Perfluorooctyl Triethoxysilane, Polyperfluoromethylisopropyl Ether).

In a preferred embodiment, the coating product comprises
a binder at a level of 15 to 30% solids by weight of the composition, preferably incorporated as an aqueous dispersion at a level of 40 to 85% by weight of the composition when provided as a 35% by solids dispersion in water,
encapsulated liquid crystal at a level of 6 to 30% solids based on the composition, preferably included as a 40% slurry, that is from 15% to 75% by weight of the composition when incorporated as a 40% slurry dispersion in water,
a dark colourant at a level of 0.1 to 10%,
a preservative at a level of 0.05 to 5%,
a wetting agent or viscosity modifier at level of 0.1 to 5%,
a UV stabiliser at a level of 0.5 to 20%,
a rheology modifier at a level of 0.01 to 5%
and an aqueous solvent at a level of 20 to 70%

The nail product is suitably in the form of a coatable liquid which may be applied to the surface using a brush or applicator to apply the liquid to the surface in an even coat. Where the nail product is formulated as a spray, the composition may be dispensed from a non-pressurised container or from a pressurised container using a propellant. Any known propellants may be used which are suitable for the intended application, for example butane, carbon dioxide, ethane and dimethyl carbonate.

The invention is illustrated by the following non-limiting examples. Percentage and part quantities are by weight based on the composition of the invention unless otherwise stated.

### EXAMPLES

A number of coating products according to the invention were prepared according to the compositions set out in Table 1 below. The compositions were prepared by mixing the components together. The encapsulated liquid crystal was a 40% aqueous slurry and the water in the slurry provided the aqueous component of the composition.

TABLE 1

| | |
|---|---|
| Component | % by weight |

Example 1

| | |
|---|---|
| Carfil 9235 | 55 |
| Liquid crystal slurry | 40 |
| (40% solids) | |
| Carfil 9241 | 1 |
| Carfil 9240 | 2 |
| Carbon black | 0.5 |
| Germaben II | 1 |

Example 2

| | |
|---|---|
| Carfil 9235 | 55 |
| Liquid crystal slurry | 40 |
| (added as 40% solids) | |
| Carfil 9241 | 1 |
| Carfil 9240 | 2 |
| Germaben II | 1 |

Example 3

| | |
|---|---|
| Syntran 5620CG | 60 |
| Hexylene glycol | 5 |
| Worlee micromer C50/25 | 1 |
| Liquid crystal slurry | 30 |
| (added as 40% solids) | |
| Germaben II | 1 |

Example 4

| | |
|---|---|
| Carfil 9235 | 50 |
| Liquid crystal 285 C. | 40 |
| (added as 40% solids) | |
| Carfil 9241 | 1 |
| Carfil 9240 | 2 |
| Germaben II | 1 |
| Benzophenone-4 | 5 |

Example 5

| | |
|---|---|
| Syntran 5620CG | 50 |
| Carfil 9241 | 0.5 |
| Hexylene glycol | 5 |
| Worlee micromer C50/25 | 1 |
| Carbon black | 0.5 |
| Liquid crystal slurry | 40 |
| (40% solids) | |
| Germaben II | 1 |

CARFIL9235 is an aqueous dispersion of polyurethane-2 comprising 34-36% solids and having a viscosity of <500 mPa·s at 25° C.

CARFIL 9240 is an aqueous dispersion of polyurethane-2 comprising 25% solids and having a viscosity of 15000 to 20000 mPa·s at 25° C.

WorleeAquaNail is a polyacrylate dispersion comprising Aqua, Polyacrylate-42, Acetyl Tributyl Citrate, Dibutyl Sebacate, Phenylpropanol, Caprylyl Glycol, Decylene Glycol

The product of each of Examples 1 to 5 was evenly applied to nails using a standard nail varnish applicator and allowed to dry. The products were then observed and tested for durability by chipping with a pencil leads of different hardness up to a hardness of 2H.

The product of Example 1 showed excellent colour brightness when applied by brush to a nail and visibly observed. The products of Examples 2 to 4 showed similarly excellent colour brightness and had improved evenness of coating. The composition of Example 3 provided excellent brightness and durability and resistance to chipping. The composition of Example 5 provided excellent brightness and adequate durability, being resistant to chipping with a pencil of hardness H, although less durable than the products of Examples 1 to 4.

Adhesion of the coating to the nail and chipping was tested using a procedure similar to the Scotch Tape test employed in adhesive coatings ASTM Test D3359-09. All the compositions applied performed satisfactorily in these tests.

1. A nail coating product comprising an aqueous chromic composition which is substantially free of organic solvent which comprises a colour-change component, a binder dispersed in an aqueous solution and a colourant dispersed or dissolved in the aqueous composition.

2. A nail coating product which is substantially free of organic solvent comprising:
i) an aqueous chromic composition comprising a colour-change component and a binder dispersed in an aqueous solution; and
ii) a separate aqueous solution comprising a colourant for use with the aqueous chromic composition to provide an underlying layer comprising colourant which underlies a layer comprising the colour-change component.

3. A nail coating product according to claim 1 wherein the colour change component comprises a thermochromic liquid crystal.

4. A nail coating product according to claim 3 wherein the colour change component comprises an encapsulated liquid crystal.

5. A nail coating product according to claim 3 wherein the colourant comprises is water soluble dye.

6. A nail coating product according to claim 3 wherein the colourant is a dark colour selected from black and blue.

7. A nail coating product according to claim 3 wherein the binder is selected from a non-ionic, hydrophobically-modified ethoxylated urethane (HEUR), an acrylic polymer, a styrene/acrylate copolymer, a styrene/acrylate/ammonium methacrylate copolymer, an epoxy resin and a polyurethane.

8. A nail coating product according to claim 3 comprising an aqueous solution containing an encapsulated liquid crystal and a binder dispersed in the aqueous solution and a dark colourant selected from black and blue provided in the said aqueous solution wherein the binder comprises at least one of a non-ionic, hydrophobically-modified ethoxylated urethane (HEUR), a polyurethane, a styrene/acrylate/ammonium methacrylate copolymer and/or an epoxy resin.

9. A nail coating product according to claim 2 which further comprises a separate aqueous top-coat composition comprising a UV absorbing agent and water.

10. A nail coating product according to claim 3 further comprising a rheology modifier.

11. A nail coating product according to claim 10 wherein the rheology modifier comprises a glycol, selected from polyethylene glycol, polypropylene glycol, polybutylene glycol and a C₂ to C₆ alkylene glycol.

12. A nail coating product according to claim 3 comprising encapsulated liquid crystal at a level of 6 to 30% solids based on the composition, a binder at a level of 15 to 30% solids by weight of the composition, a dark colourant selected from black and blue at a level of 0.1 to 10% and a preservative at a level of 0.05 to 5%.

13. A nail coating product according to claim 12 comprising at least one of a wetting agent or viscosity modifier at level of 0.1 to 5%, a UV stabiliser at a level of 0.5 to 20%, a rheology modifier at a level of 0.01 to 5%.

14. A method of providing a nail coating which comprises applying an aqueous chromic composition according claim 1 to a substrate to be coated.

15. A method according to claim 14 which comprises applying an underlying layer to the substrate, drying the underlying layer and then applying the aqueous the said aqueous chromic composition to provide a dried nail coating.

16. A method according to claim 14 further comprising applying to the dried surface of the chromic composition an aqueous top-coat composition comprising a UV absorber and drying the applied top-coat composition.

17. A method according to claim 14 wherein the substrate is selected from keratinous nails, artificial, non-keratinous nails, nail extensions, an existing coating on an underlying keratinous or artificial nail.

18. A process of providing a colourant to a nail comprising:
i) applying the aqueous chromic composition to a nail; or
ii) applying a separate aqueous solution comprising a colourant to a nail to provide a colourant layer and applying the aqueous chromic composition to the colourant layer.
